(19) United States
(12) Patent Application Publication
(10) Pub. No.: US 2007/0173825 A1
(43) Pub. Date: Jul. 26, 2007

(54) Spinal rod parallel coupler

(75) Inventors: Amir Ali Sharifi-Mehr, Bloomingdale, NJ (US); Charles L. Bush JR., Fairfield, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Spine, Cestas (FR)

(21) Appl. No.: 11/335,987

(22) Filed: Jan. 20, 2006

(51) Int. Cl. A61F 2/30

(52) U.S. Cl. 606/61

(57) ABSTRACT

A spinal rod coupler used for joining spinal rods in a parallel fashion. The coupler is adapted to join rods of different thicknesses or flexibilities while occupying a minimal amount of volume inside a patient. The coupler preferably has a V-shaped channel adapted to receive the spinal rods such that the spinal rods make a line contact with the V-shaped channel at two points in cross section. The coupler also has at least one blocker adapted to exert force against the spinal rod or rods in order to hold the spinal rod in the coupler.

BACKGROUND OF THE INVENTION

The present invention relates to a vertebral osteosynthesis device that can be used to brace a spine, for example, following accidental fracture, or to strengthen or brace a deviated spine, as in the case of scoliosis or kyphosis. More particularly, this invention is related to a novel connector which fixes the location and enhances the flexibility of spinal rod systems.

As is known in the art, spinal rods are commonly used to stabilize segments of the spine. These spinal rods are typically attached to vertebrae using set screws. These rods are shaped to adapt to the curve of the spine. Different lengths of spinal rods are used to correct different portions of the spine.

Pedicle screws for affixing spinal rods to vertebrae are known in the art, for example, U.S. Pat. No. 6,261,287 discloses apparatus for bracing vertebrae. These devices typically include a fastener having a screw portion and a screw head, a coupling element or tulip which receives the screw head and a spinal rod, and a fixation set screw or nut which fixes the spinal rod, fastener and coupling element. As disclosed in the '287 patent, the fastener can be in polyaxial relationship with the coupling element or tulip, or the tulip can be the head of the fastener with a fixed relationship. When spinal fixation is desired, fasteners are typically threaded into or hooked onto the vertebrae of the area to be braced. A spinal rod is then shaped appropriately to hold the vertebrae in place. The spinal rod is then coaxed into and fixed within the coupling elements of the pedicle screws. Thus, the spinal rod serves to stabilize a region of the spine.

It is often desirable to utilize multiple spinal rods connecting vertebrae on one or both sides of the spine. The multiple rods may have different thicknesses or flexibilities in order to support different portions of the spine. For example, a portion of the spine that is only slightly damaged may be supported by a thin, slightly flexible spinal rod while adjacent vertebrae that are more damaged may require a thicker, more rigid spinal rod for support. It is therefore necessary to join parallel or similarly aligned spinal rods having different diameters and characteristics to each other in an abutting or adjacent fashion.

Thus, there exists a need for a parallel spinal rod coupler which joins two spinal rods which may have differing diameters in a manner that does not compromise the integrity of the rod system and minimizes occupied space.

### SUMMARY OF THE INVENTION

The present invention provides a coupler which joins two spinal rods, preferably in a parallel manner.

In one embodiment, the present invention is a spinal rod coupler comprising a coupler body having a channel, the channel having a at a first end adapted to receive a spinal rod therethrough, and a first blocker associated with the coupler body such that said blocker being moveable between a first position into which a spinal rod can be moved axially in said channel to a second position at which a force is imparted on the spinal rod when the spinal rod is in the channel, wherein the channel has a generally V-shaped portion generally opposite the blocker and adapted to receive the spinal rod such that the spinal rod makes a line contact with the V-shaped portion at two points in cross section.

In a further embodiment, the channel may have a second open end adapted to receive a spinal rod therethrough, and the coupler may further comprise a second blocker associated with the coupler body such that said second blocker is moveable from a first position to a second position to input force on the spinal rod when the spinal rod is in the channel. In this embodiment, the coupler may also comprise a a center post adapted to be inserted into the coupler to bisect the channel into the first end and the second end. The channel may have a different diameter at the first end and the second end.

In one embodiment, the first blocker or the second blocker may be a set screw. In some embodiments, both blockers may comprise set screws.

In one embodiment, the first blocker is rotatable between the first position and the second position. In some embodiments, at least one of the first blocker and second blocker is rotatable between the first position and second position.

In an alternative embodiment, the present invention is a spinal rod coupler comprising a coupler body having a first channel with a V-shaped portion and a second channel having a V-shaped portion adjacent to the first channel and parallel with the first channel, the first channel and the second channel adapted to receive spinal rods, a first blocker associated with the first channel such that the first blocker is moveable from a first position to a second position to exert a force on a first spinal rod in the first channel, and a second blocker associated with the second channel such that the second blocker is moveable from a first position to a second position to exert a force on a second spinal rod in the second channel.

In one embodiment, at least one of the first blocker or the second blocker may be a set screw.

In one embodiment, the first channel may have a different diameter than the second channel.

In another embodiment, the present invention is a method of joining two spinal rods, comprising providing a coupler having a partially V-shaped channel extending through the coupler and two blockers adapted to extend into the channel, inserting a first spinal rod into the first end of the channel such that the first spinal rod makes a line contact with the V-shaped portion at two points in cross section, inserting a second spinal rod into the second end of the channel the such that the second spinal rod makes a line contact with the V-shaped portion at two points in cross section, and moving the two blockers from a first position to a second position such that the blockers press the first and second spinal rods into the v-shaped portion of the channel.

### BRIEF DESCRIPTION OF THE DRAWINGS

FIG. 1 is a perspective view of a coupler according to a preferred embodiment of the invention for coupling two spinal rods aligned in a similar direction.

FIG. 2 is a cross-section view of the coupler depicted in FIG. 1.

FIG. 3A is a top view of the coupler depicted in FIG. 1.

FIG. 3B is a cross-section view of the coupler depicted in FIG. 3A.

FIG. 4A is an alternate embodiment of a coupler according to the present invention adapted for connecting adjacent spinal rods aligned in a similar direction.

FIG. 4B is a cross-section view of the coupler depicted in FIG. 4A.

### DETAILED DESCRIPTION OF THE PREFERRED EMBODIMENT

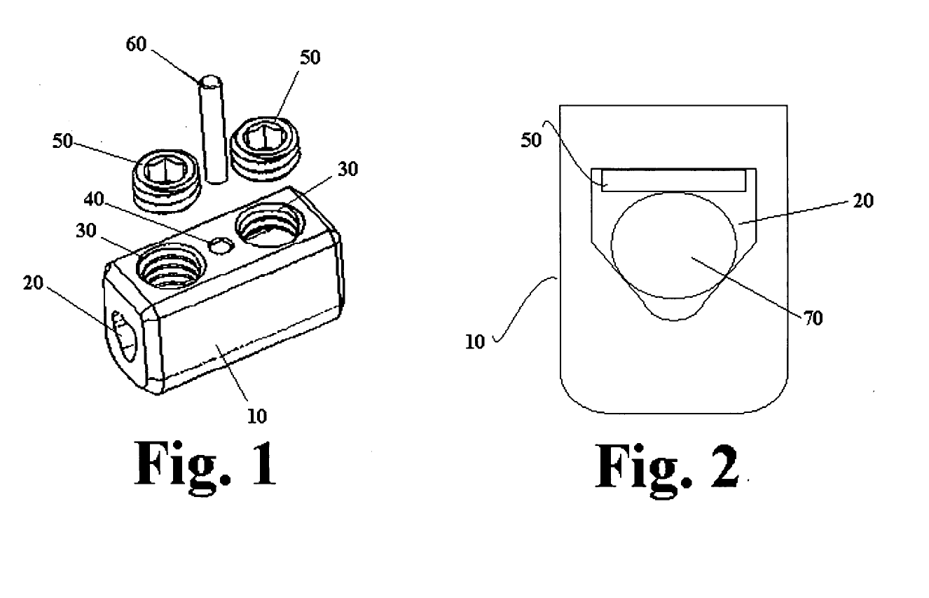

The present invention will now be described with reference to the figures. FIG. 1 depicts a perspective view of a coupler 10 according to a preferred embodiment of the present invention. The coupler 10 has a rod-receiving hole 20 on each end. The rod-receiving holes 20 are adapted to receive spinal rods of varying sizes. The coupler 10 also has set screw holes 30 adapted to receive set screws 50, which includes threads as on a conventional screw. The set screw holes 30 are in this embodiment threaded and extend to the rod-receiving channel 20. The set screws may be adapted to be turned with a torx wrench or a screwdriver. The set screws 50 are inserted into the set screw holes 30, preferably before the start of the surgical procedure in which the coupler is to be inserted. The coupler 10 may also have a post-receiving hole 40. The post-receiving hole 40 is preferably located in the longitudinal center of the coupler. The post-receiving hole 40 is adapted to receive a center post 60 therein.

The center post 60 preferably extends through the rod-receiving channel 20 to the bottom of the channel. The center post 16 is in the disclosed embodiment press fit into the coupler 10 forming an interference fit. One skilled in the art would recognize that there are many other ways to affix the center post, for example, the post may be threaded and screwed in to the coupler 10 or set in place with a biocompatible adhesive in alternative embodiments. In yet still further embodiments, the center post may be integral with the coupler 10. One skilled in the art would recognize that the center post may be replaced with an alternate method of preventing spinal rods from slipping, such as an integral center wall.

FIG. 2 depicts a cross-section view of the coupler 10. The coupler 10 shown in FIG. 2 is depicted with a spinal rod 70 inserted into the coupler 10. The cross-section also depicts the bottom of a set screw 50 protruding into the rod-receiving channel 20. The bottom of the set screw 50 applies pressure to the spinal rod 70 to lock the spinal rod 70 into the coupler 10. The rod-receiving channel 20 preferably has a V-shaped recess. This V-shaped recess serves to lock the spinal rod 70 to the coupler 10. The V-shaped recess is preferable because it serves to lock spinal rods having various different diameters into the rod-receiving channel 20.

In practice, a spinal rod which may already be inserted into pedicle screws or other means of holding a spinal rod is inserted into the coupler at rod-receiving channel 20. The rod is preferably inserted into the coupler until it makes contact with the center post 60. The center post prevents the rod from extending into the channel 20 portion designated for the second spinal rod. After the spinal rod is inserted into the channel 20, the set screw 50 above the channel 20 where the spinal rod was inserted is rotated. This causes the set screw 50 to move downward within the coupler 10 toward the rod 70. The set screw 50 then makes contact with the rod 70 and presses the rod against the V-shaped sides of the channel 20 as seen in FIG. 2. The rod is held in place by contact in three points: the set screw and the two sides of the V-shaped channel. A second spinal rod is then inserted into the opposite end of the coupler 10 at the rod-receiving channel 20. The second set screw 50 is then rotated downwardly to make contact with the second spinal rod.

The three-point contact of the coupler increases the resistance to axial pull-out forces. As a result of this increased holding strength created by the V-shaped channel, the length and overall size of the coupler can be reduced which is advantageous in spinal surgery because of the lack of space to be used for spinal fixation apparatus.

The various components of the invention are preferably made from a biologically inert material, for example, any metal customarily used for surgical devices and particularly those used for bone screws and spinal rods, such as titanium or stainless steel. Other suitable materials include, but are not limited to, alloys, composite materials, ceramics or carbon fiber materials.

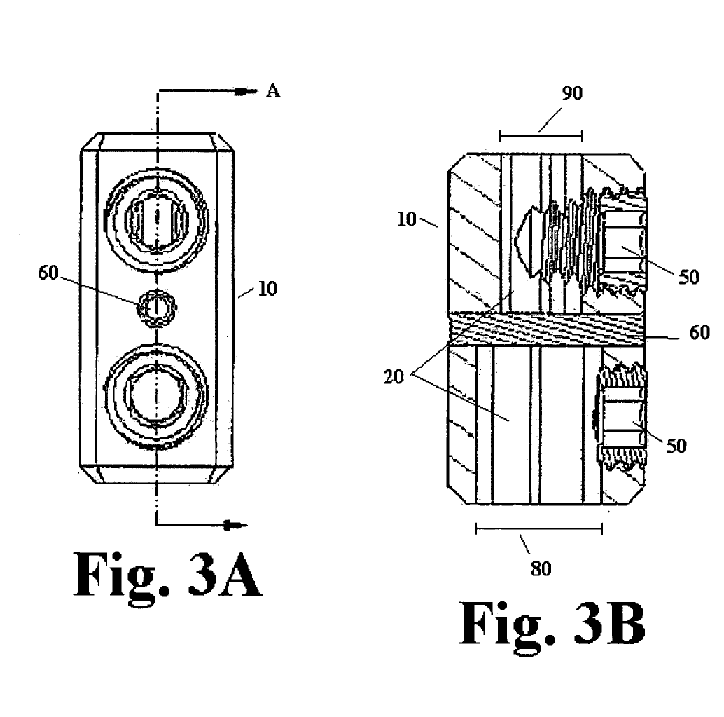

FIG. 3A shows a top view of one embodiment of a coupler 10 according to the present invention. FIG. 3B depicts a cross-section view of the coupler depicted in FIG. 3A at axis A. The cross-section view shows the coupler 10 having rod-receiving channels 20 of different heights. Although the coupler 10 is adapted to receive spinal rods of varying diameters, a coupler 10 may be provided with channels 20 of different heights to accept spinal rods having drastically different diameters. The height 90 depicts a rod-receiving channel which is narrower than the height 80. These different heights are adapted to receive spinal rods having different diameters. The coupler 10 also has a center post 60 extending through the coupler 10. Two different set screws 50 are depicted in the cross-section view of FIG. 3B. Set screw 50 which extends into channel 90 is preferably longer because it interacts with a narrower spinal rod. Set screw 50 which interacts with channel 80 is preferably a shorter set screw that extends only partially into the channel 80 because it is adapted to interact with a thicker spinal rod.

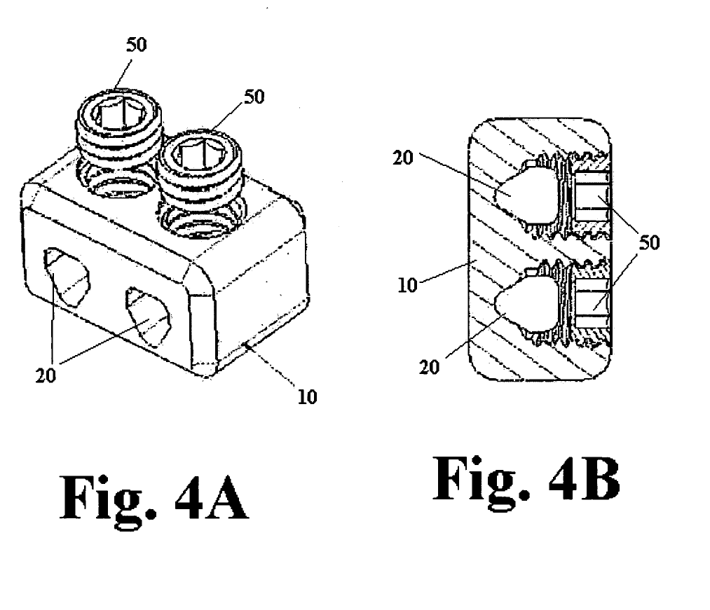

An alternate embodiment of a spinal rod coupler is depicted in FIGS. 4A and 4B. FIG. 4A is a perspective view of a coupler according to the present invention. The coupler 10 is shown with adjacent rod-receiving channels 20 for joining adjacent rods that are oriented in the same direction. The rod-receiving channels 20 preferably have a V-shape. The coupler 10 also has two set screws 50 which extend into the channels 20. FIG. 4B is a cross-section of the coupler depicted in FIG. 4A. The coupler 10 has set screws 50 which extend into the V-shaped channels 20. This embodiment is useful if spinal rods are to be joined in an adjacent fashion.

Although the above embodiments have been described with reference to set screws having continuous threads, for purposes of holding the rods in place, one skilled in the art would recognize that numerous other expedients for holding the spinal rods to the coupler may be used. One such device, a set screw having flanges which are like threads, is disclosed in U.S. Pat. No. 6,565,565 to Yuan et al. which is hereby incorporated by reference. One skilled in the art would recognize that the device disclosed in Yuan would be useful for locking the spinal rods to the coupler according to the present invention. Other devices might be moveably engaged within the body of the coupler in a linear fashion as opposed to in a rotatable fashion. An example of such a device is a blocker that has an interference fit within the body and can be moved towards the spinal rod to impose a force on it.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A spinal rod coupler comprising:
a coupler body having a channel, said channel having a at a first end adapted to receive a spinal rod therethrough; and
a first blocker associated with the coupler body such that said blocker being moveable between a first position into which a spinal rod can be moved axially in said channel to a second position at which a force is imparted on the spinal rod when the spinal rod is in the channel;
wherein the channel has a generally V-shaped portion generally opposite the blocker and adapted to receive the spinal rod such that the spinal rod makes a line contact with the V-shaped portion at two points in cross section.

2. The coupler of claim 1, wherein the channel has a second open end adapted to receive a spinal rod therethrough, and the coupler further comprises a second blocker associated with the coupler body such that said second blocker is moveable from a first position to a second position to input force on the spinal rod when the spinal rod is in the channel.

3. The coupler of claim 2 further comprising a center post adapted to be inserted into the coupler to bisect the channel into the first end and the second end.

4. The coupler of claim 2 wherein the channel has a different diameter at the first end and the second end.

5. The coupler of claim 1 wherein the first blocker comprises a set screw.

6. The coupler of claim 2 wherein at least one of the first blocker and the second blocker comprises a set screw.

7. The coupler of claim 1 wherein the first blocker is rotatable between the first position and the second position.

8. The coupler of claim 2, wherein at least one of the first blocker and second blocker is rotatable between the first position and second position.

9. The coupler of claim 2, further including a spinal rod.

10. A spinal rod coupler comprising:
a coupler body having a first channel with a V-shaped portion and a second channel having a V-shaped portion adjacent to the first channel and parallel with the first channel, the first channel and the second channel adapted to receive spinal rods;
a first blocker associated with the first channel such that the first blocker is moveable from a first position to a second position to exert a force on a first spinal rod in the first channel; and
a second blocker associated with the second channel such that the second blocker is moveable from a first position to a second position to exert a force on a second spinal rod in the second channel.

11. The coupler of claim 7, wherein the first blocker is a set screw.

12. The coupler of claim 8, wherein the second blocker is a set screw.

13. The coupler of claim 7, wherein the first channel has a different diameter than the second channel.

14. A method of joining two spinal rods, comprising:
providing a coupler having a partially V-shaped channel extending through the coupler and two blockers adapted to extend into the channel,
inserting a first spinal rod into the first end of the channel such that the first spinal rod makes a line contact with the V-shaped portion at two points in cross section,
inserting a second spinal rod into the second end of the channel the such that the second spinal rod makes a line contact with the V-shaped portion at two points in cross section, and
moving the two blockers from a first position to a second position such that the blockers press the first and second spinal rods into the v-shaped portion of the channel.
